(19) United States
(12) Patent Application Publication
(10) Pub. No.: US 2026/0053463 A1
(43) Pub. Date: Feb. 26, 2026

(54) Piezoelectric Micromachined Ultrasonic Transducers for Blood Pressure Monitoring

(71) Applicant: The Regents of the University of California, Oakland, CA (US)

(72) Inventors: Yande Peng, Berkeley, CA (US); Pan Xia, Berkeley, CA (US); Liwei Lin, Berkeley, CA (US); Wei Yue, Berkeley, CA (US)

(73) Assignee: The Regents of the University of California, Oakland, CA (US)

(21) Appl. No.: 19/060,770

(22) Filed: Feb. 23, 2025

Related U.S. Application Data
(63) Continuation of application No. PCT/US23/74977, filed on Sep. 24, 2023.
(60) Provisional application No. 63/410,152, filed on Sep. 26, 2022.

(51) Int. Cl. A61B 8/04; A61B 8/00; B06B 1/06

(52) U.S. Cl. CPC A61B 8/04; A61B 8/4227; A61B 8/4472; A61B 8/4488; B06B 1/0622

(57) ABSTRACT

An array of piezoelectric micromachined ultrasonic transducers is used for blood pressure monitoring.

### CROSS-REFERENCES TO RELATED APPLICATIONS

This application is a continuation of PCT/US23/74977, filed: Sep. 24, 2023, which claims priority to U.S. Provisional Application No. 63/410,152, filed: Sep. 26, 2022, the disclosures of which are hereby incorporated by reference in its entirety for all purposes.

### INTRODUCTION

Hypertension has contributed significantly to cardiovascular disease and has triggered billions medical expenses in the public health care every year^{[1]}. High blood pressure is often called the silent killer since there are no abnormal symptoms in the early stage of the illness. Early medical intervention can effectively lower the risk of hypertension^{[2]} and reduce the health care cost; however, there are currently no suitable monitoring systems.

### SUMMARY OF THE INVENTION

We disclose a real-time blood pressure (BP) monitoring scheme based on piezoelectric micromachined ultrasonic transducers (PMUT). The system may include both an active sensor and the related analog front-end (AFE) for wireless communications to record and identify abnormal behaviors in real time. Acoustic beamforming and line-scanning methods are disclosed to increase the stability and signal strength from various noise sources. PMUT designs, fabrications and testing results are disclosed as proof-of-concept. Preliminary bench experiments and phantom measurements are disclosed which have validated the potential of such technology as a monitoring solution for cardiovascular health.

In an aspect the invention provides a blood pressure monitoring device comprising an array of piezoelectric micromachined ultrasonic transducers (PMUTs) for blood pressure monitoring substantially as disclosed herein.

In an aspect the invention provides a method comprising monitoring blood pressure with an array of piezoelectric micromachined ultrasonic transducers (PMUTs) substantially as disclosed herein.

The invention provides embodiments disclosed herein, including devices:
further comprising both an active sensor and a related analog front-end (AFE) for wireless communications, configured to record and identify abnormal behaviors in real time;
configured for acoustic beamforming and line-scanning, so as to increase the stability and signal strength from noise sources;
wherein beamforming technology is utilized to optimize ultrasonic energy, and signals measured by the PMUT sensor are analyzed and post-processed in an analog front-end (AFE);
configured as a wearable system is composed of the PMUT array in a flexible substrate as the sensor, wherein beamforming optimizes ultrasonic energy, and signals measured by the PMUT sensor are analyzed and post-processed in an analog front-end (AFE) and transmitted to a portable device (such as a cell phone) to monitor their blood pressure in real time, as shown in FIG. 1;
configured as a 31×35 array design and a total sensor size of 5 mm by 5 mm, wherein the radius of each element is 29 μm on a two 1-μm-thick AlN bimorph and dual-electrode diaphragm to have a designed frequency of 6 MHz in liquid, and the array structure has 20 independent channels for the purpose of beamforming, as shown in FIGS. 2A-C;
configured as follows: a 200-nm seed AlN layer is first deposited by the AC sputtering process for good crystallinity, followed by the bottom Mo/bottom AlN/mid Mo depositions with the thickness of 150 nm, 1 μm and 150 nm, respectively; the middle Mo layer is then patterned, and a 1-μm thick top AlN layer and a 150-nm thick Mo electrode layer are deposited; the AlN layers are then patterned via the reactive ion etching process, wherein the diaphragm size is defined by a backside DRIE process, and The PMUT sensors are then connected to the outside circuit through wire bonding using an operation scheme, as shown in FIGS. 3A-F;
configured to deploy beamforming and use the phase-delay on elements/channels in the array such that the signal emitted by different elements/channels can be in phase at the focal points/lines by adding up the acoustic pressure, wherein different phase delays are applied to the system by adjusting the phase in PMUT sensors locating in different positions, as shown in FIGS. 4A-C;
configured to addresses the shift of the artery and sensor positions due to the motion of muscles/tissues, wherein the main reflected acoustic beam of the cylindrical artery comes from the path that follows the law of reflection; however, the main reflection path may change correspondingly if the artery shifts, as shown in FIGS. 5A-B;
configured for a two-step method combining the adjustment and line-scanning beamforming method using each measurement to obtain the high signal outputs, wherein the transmitted arrays are controlled with different phases to achieve beamforming effect along the pre-defined path and the largest signal collected is used for the post-processing, to obtain signals coming from the right path and maintain the good signal-to-noise ratio;
comprising a horizontal section comprising multiple individually controlled arrays, wherein depending on the relative position of the artery, each array will receive distinct echo signals, wherein this information is used to fine-tune the device's position within the range where the primary vertical detection array, can precisely measure the required metrics, facilitated by beamforming techniques, as shown in FIG. 6;
bonded to a printed circuit board (PCB) and comprising a defined pattern of a dual-electrode design providing crystallinity of the AlN with a pillar-like morphology, as shown in FIGS. 7A-D; and/or
integrated into a non-invasive wearable form; particularly wherein the PMUT sensor is discreetly embedded beneath a 3D-printed wearable mold, simulating the form factor of a typical smartwatch, the sensor array is positioned in proximity to the radial artery, maintaining reliable contact with a person through a watch band.

In aspects the invention provides a method comprising monitoring blood pressure in real time with a device herein.

In embodiments, beamforming technology is utilized to optimize ultrasonic energy, and signals measured by the PMUT sensor are analyzed and post-processed in an analog front-end (AFE).

The invention encompasses all combinations of the particular embodiments recited herein, as if each combination had been laboriously recited.

### BRIEF DESCRIPTION OF THE DRAWINGS

FIG. 1. Blood pressure sensor through beamforming.

FIG. 2A. PMUT design with a 31×35 array design as an example with a total sensor size of 5 mm by 5 mm. FIG. 2B. The radius of each element is 29 μm on a two 1-μm-thick AlN bimorph and dual-electrode diaphragm. FIG. 2C. The array structure has 20 independent channels.

FIG. 3A. PMUT fabrication process with a seed AlN layer deposited, followed by bottom Mo/bottom AlN/mid Mo depositions. FIG. 3B. The middle Mo layer is then patterned. FIG. 3C. A thick top AlN layer and a thick Mo electrode layer are deposited. FIG. 3D. The AlN layers are then patterned. FIG. 3E. The diaphragm size is defined by a backside DRIE process. FIG. 3F. The PMUT sensors are then connected to the outside circuit through wire bonding per an operation scheme.

FIG. 4A. Simulation performance improvement by the beamforming. FIG. 4B. different phase delays are applied by adjusting the phase in PMUT sensors locating in different positions. FIG. 4C. Numeric analysis as well as simulation (FIG. 4A) show stronger output pressure with the beamforming scheme.

FIG. 5A. The main reflected acoustic beam of the cylindrical artery. FIG. 5B. The main reflection path may change correspondingly if the artery shifts.

FIG. 6. This chip includes a horizontal section of multiple individually controlled arrays, as highlighted in red in the detailed zoom-in images on the right. The primary vertical detection array is highlighted in blue on the left.

FIG. 7A. Optical image of a fabricated device bonded to our printed circuit board (PCB). FIG. 7B. Zoom-in figure. FIG. 7C. The cross-section of the fabricated device and the etched cavity size. FIG. 7D. Zoom-in figure in shows good crystallinity with a pillar-like morphology.

FIG. 8A. Experimental setup with the PMUTs in deionized water; a silicone tube is placed above the sensor. FIG. 8B. Receiving signals show four clear peaks.

FIG. 9A. A mechanical pump to circulate imitating blood fluid to the arm. FIG. 9B. An arm phantom. FIG. 9C. PMUT sensors are mounted on the arm; signals are collected through the oscilloscope after being amplified.

FIG. 10A and FIG. 10B. Echo signals collected from the phantom measurements are plotted.

FIG. 11. Diameter variation over time.

FIG. 12A. The PMUT sensor is embedded beneath a 3D-printed wearable mold in the form factor of a smartwatch. FIG. 12B. The echo signals from the radial arteries can be clearly identified. FIG. 12C. The blood pressure waveform of the volunteer.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

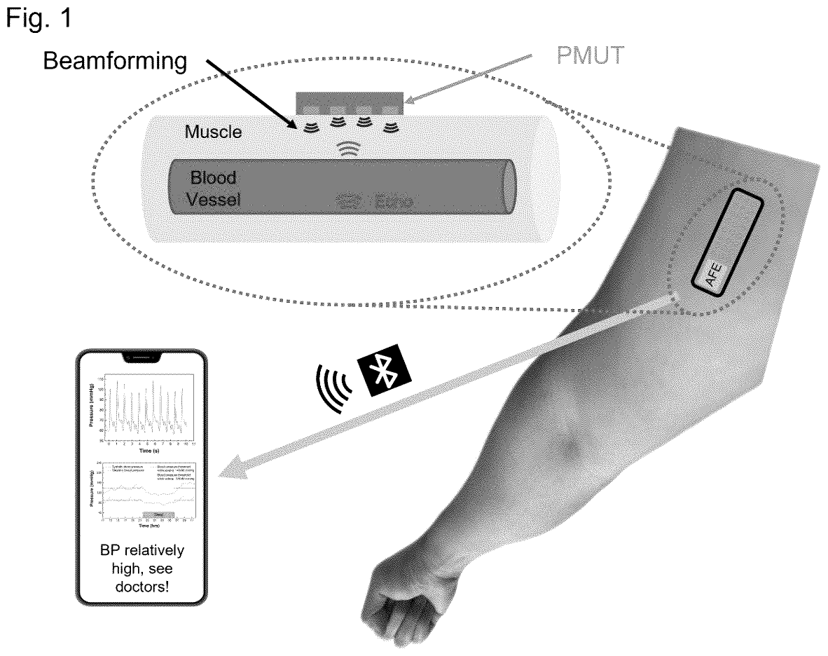

FIG. 1 shows a disclosed blood pressure sensor through beamforming. The disclosed wearable system is composed of the PMUT array in a flexible substrate as the sensor. Beamforming technology is disclosed to optimize ultrasonic energy. In this case, signals measured by the PMUT sensor are analyzed and post-processed in the analog front-end (AFE) and transmitted to the portable device (such as a cell phone) through Bluetooth to monitor their blood pressure in real time. The working principle of the PMUT sensor for blood pressure measurements is based on the artery diameter variations due to the corresponding BP^{[3]};

$$p ( t ) = {p}_{d} \times {e}^{β ( \frac{D ( t )}{{D}_{d}} - 1 )}$$

Where the p_{d}, D_{d}, and β are the diastolic pressure (which can be calibrated externally), diastolic arterial diameter and vessel stiffness, respectively. The BP value is calculated via the artery diameter D_{d}, which is measured by a pulse-echo measurement (ultrasonic waves can generate echoes at the artery-blood interface due to the varying acoustic impedance and the time-of-flight interval between the echoes is used to characterize the diameter of the artery).

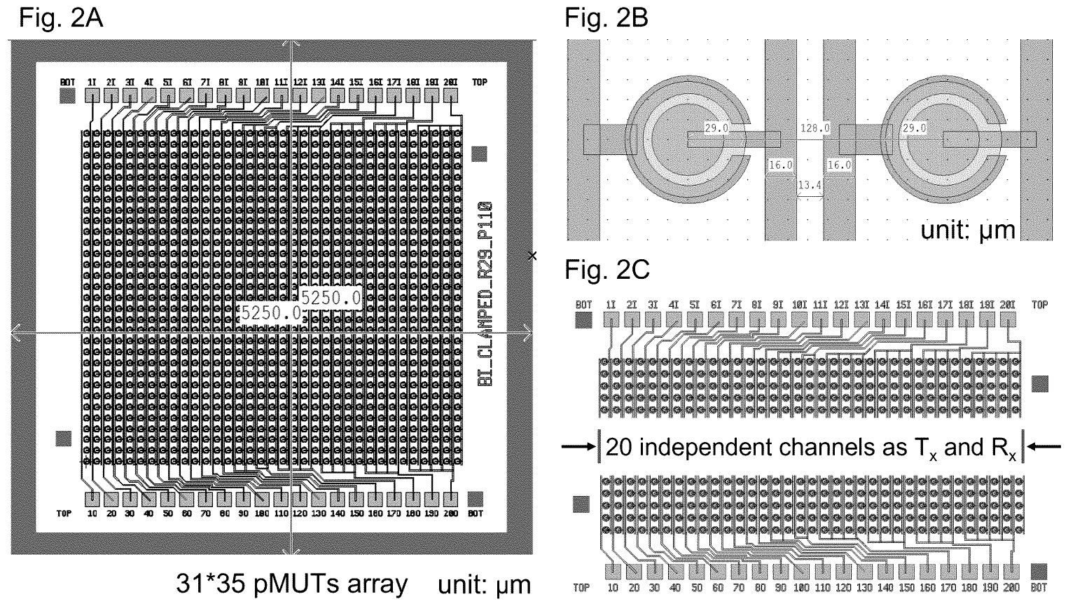

One PMUT design is illustrated in FIGS. 2A-C with a 31×35 array design as an example with a total sensor size of 5 mm by 5 mm (FIG. 2A). Due to the small size of the PMUT, many individual PMUT units are designed in the array format to increase the acoustic pressure and signal-to-noise ratio. On the other hand, the overall chip size is to maintain a small form-factor to be placed near-by the blood vessel for blood pressure detections. In the prototype design, the radius of each element is 29 μm on a two 1-μm-thick AlN bimorph and dual-electrode diaphragm^{[4]} to have a designed frequency of 6 MHz in liquid (FIG. 2B). The array structure has 20 independent channels for the purpose of beamforming (FIG. 2C).

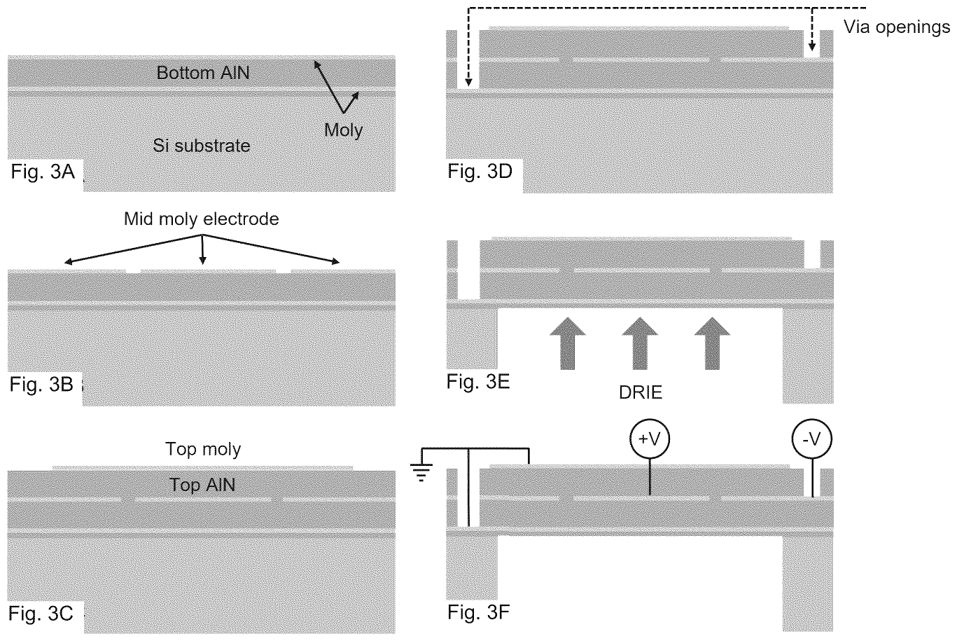

A fabrication process flow of the disclosed PMUT is shown in FIGS. 3A-F. A 200-nm seed AlN layer is first deposited by the AC sputtering process for good crystallinity, followed by the bottom Mo/bottom AlN/mid Mo depositions with the thickness of 150 nm, 1 μm and 150 nm, respectively (FIG. 3A). The middle Mo layer is then patterned (FIG. 3B) and a 1-μm thick top AlN layer and a 150-nm thick Mo electrode layer are deposited (FIG. 3C). The AlN layers are then patterned via the reactive ion etching process (FIG. 3D). Finally, the diaphragm size is defined by a backside DRIE process (FIG. 3E). The PMUT sensors are then connected to the outside circuit through wire bonding and the operation scheme is shown in FIG. 3F.

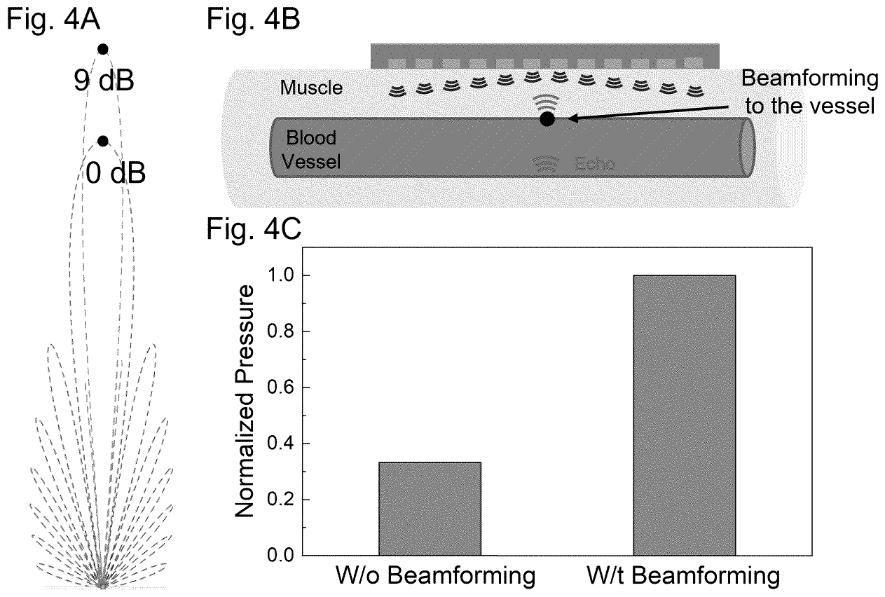

Beamforming has been widely used in radio frequency (RF) communication systems as a method to boost the signal. The idea is to use the phase-delay on elements/channels in the array such that the signal emitted by different elements/channels can be in phase at the focal points/lines by adding up the acoustic pressure. FIGS. 4A-C illustrates the performance improvement by the beamforming. As is shown in FIG. 4B, different phase delays are applied to the system by adjusting the phase in PMUT sensors locating in different positions. Numeric analyses as well as simulations are conducted (FIG. 4A and FIG. 4C) to show that with the beamforming scheme, the output pressure can be ˜3 times stronger than that of the same system without beamforming.

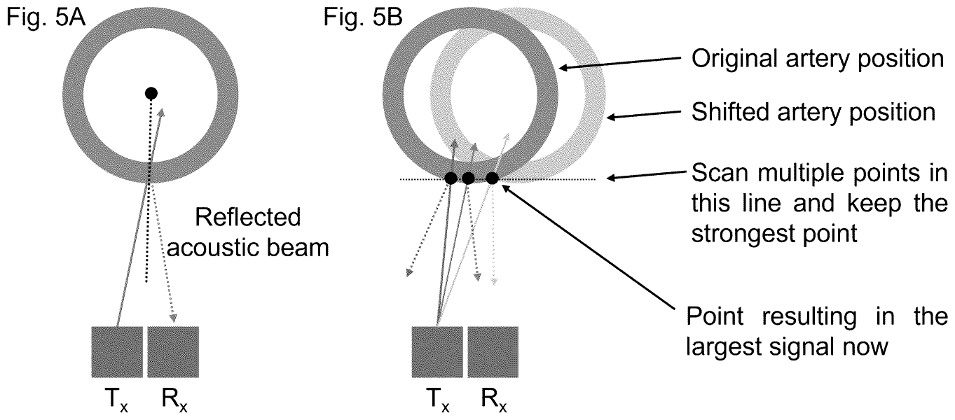

Another practical problem the invention addresses the shift of the artery and sensor positions due to the motion of muscles/tissues. For example, FIG. 5A shows the main reflected acoustic beam of the cylindrical artery should come from the path that follows the law of reflection. However, the main reflection path may change correspondingly if the artery shifts (FIG. 5B). Therefore, a two-step method combining the adjustment and line-scanning beamforming method are disclosed for each measurement to obtain the high signal outputs. In practice, the transmitted arrays are controlled with different phases to achieve beamforming effect along the pre-defined path and the largest signal collected is used for the post-processing. As such, one can obtain signals coming from the right path and maintain the good signal-to-noise ratio.

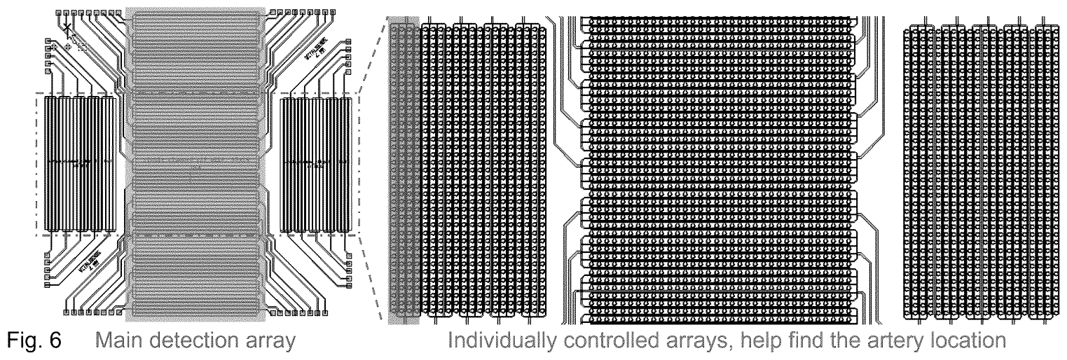

One disclosed design example can be seen in FIG. 6. The designed chip is composed of two main sections. The horizontal section consists of multiple individually controlled arrays, as highlighted in red in the detailed zoom-in images on the right. These arrays are strategically designed to assist in locating the artery. Depending on the relative position of the artery, each array will receive distinct echo signals. This information aids us in fine-tuning the device's position within the range where the primary vertical detection array, highlighted in blue on the left, can precisely measure the required metrics, facilitated by beamforming techniques.

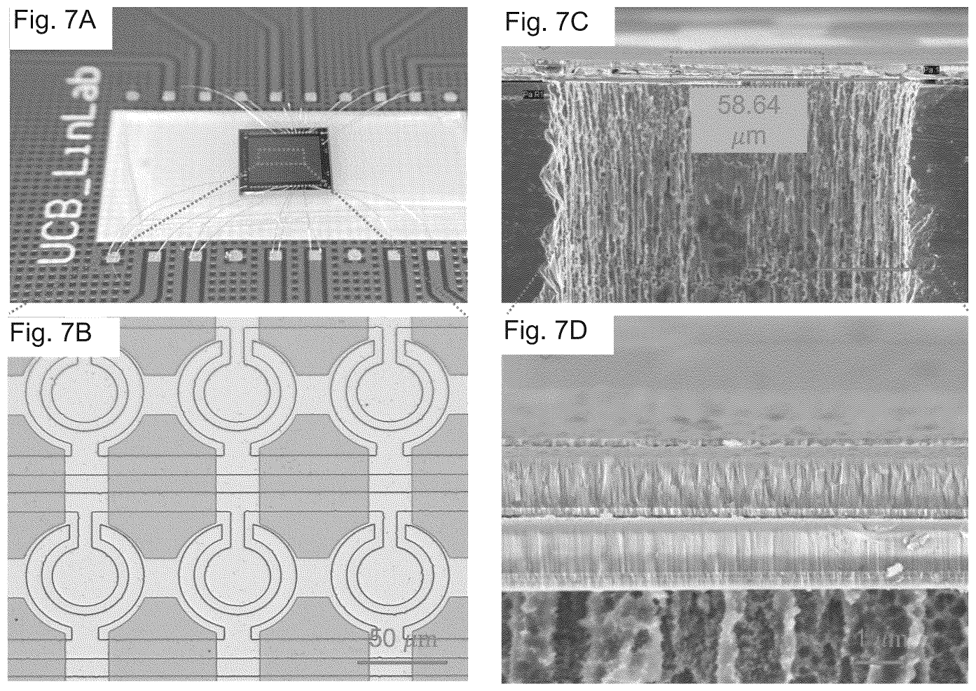

An optical image of a device example is shown in FIG. 7A where a fabricated device is bonded to our printed circuit board (PCB). The zoom-in figure can be observed in FIG. 7B and the well-defined pattern of the dual-electrode design is clearly observed. FIG. 7C shows the cross-section of the fabricated device and the etched cavity size. The zoom-in figure in FIG. 7D shows the good crystallinity of our AlN with a pillar-like morphology.

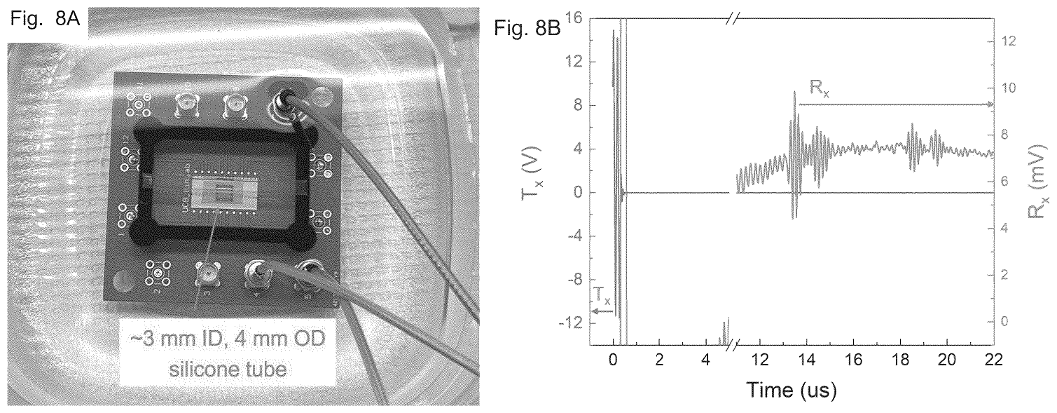

A first test is conducted by measuring the silicone tube in the experimental setup with the PMUTs in deionized water. A silicone tube with similar acoustic impedance and size (3 mm inner diameter and 4 mm outer diameter) to the real human artery is chosen as the target and placed above the sensor with a distance of 10 mm (FIG. 8A). A rectangular pulse wave is applied to the transmitted PMUT channels with an amplitude of 24 Vₚₚ. Receiving signals show four clear peaks (FIG. 8B) coming from the two sides of the anterior walls and posterior walls and the distance corresponds well with the silicone tube size.

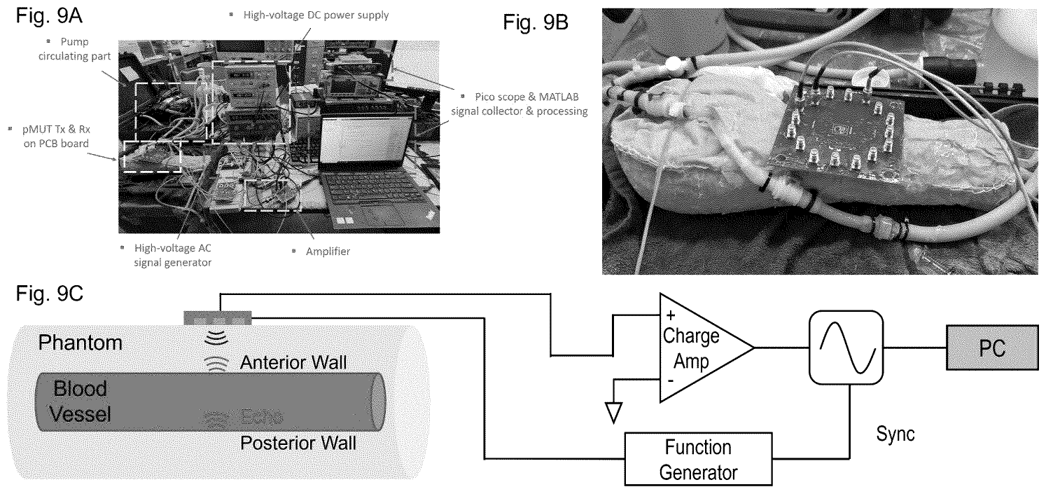

Phantom evaluations are conducted to further demonstrate the possible applications. An arm phantom is manufactured using materials close to the real tissue with the artery and vein phantom (FIG. 9B). A mechanical pump is used to circulate imitating blood fluid to the arm with the control through Arduino to imitate the pulsatile blood flow in the artery (FIG. 9A). PMUT sensors are mounted on the arm and aligned well with the artery for the diameter monitoring. Signals are collected through the oscilloscope after being amplified by a charge amplifier (FIG. 9C).

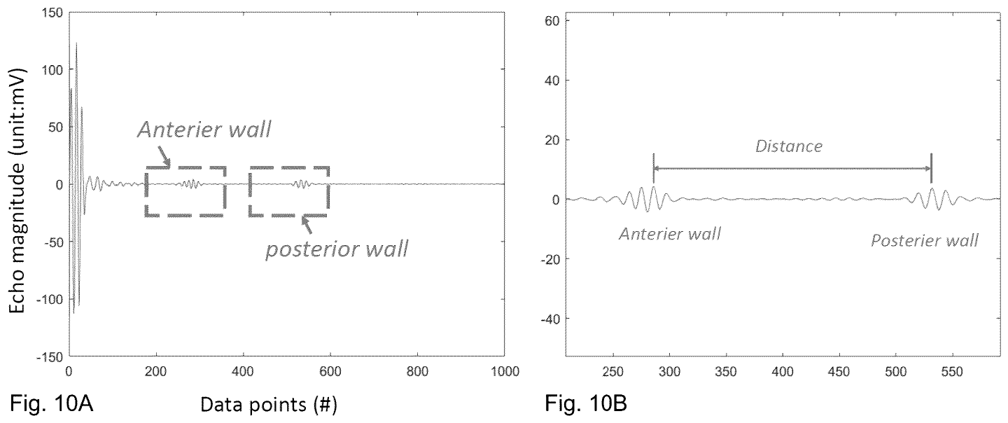

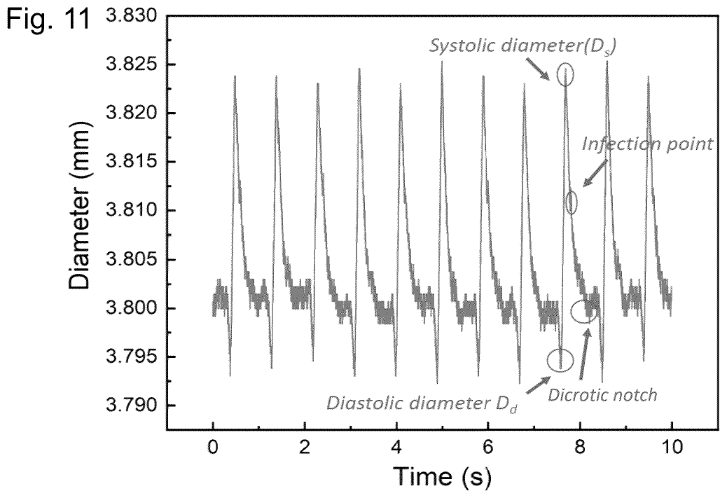

Echo signals collected from the phantom measurements are plotted as FIG. 10A and FIG. 10B. Peak values of the collected echoes are used to extract the diameter distance of that moment. Repeating this process, we can obtain the real-time diameters of the phantom arm which can be further translated to pressure values. As is shown in FIG. 11, the diameter variation over time is obtained from the measurement results. The diameter change corresponds well to the real variation in terms of frequency (˜1 Hz) and the amplitude (˜300 μm). Some detailed features of cardiovascular behaviors such as inflection point and dicrotic notch are also captured, which proves the good sensitivity of the PMUT sensors.

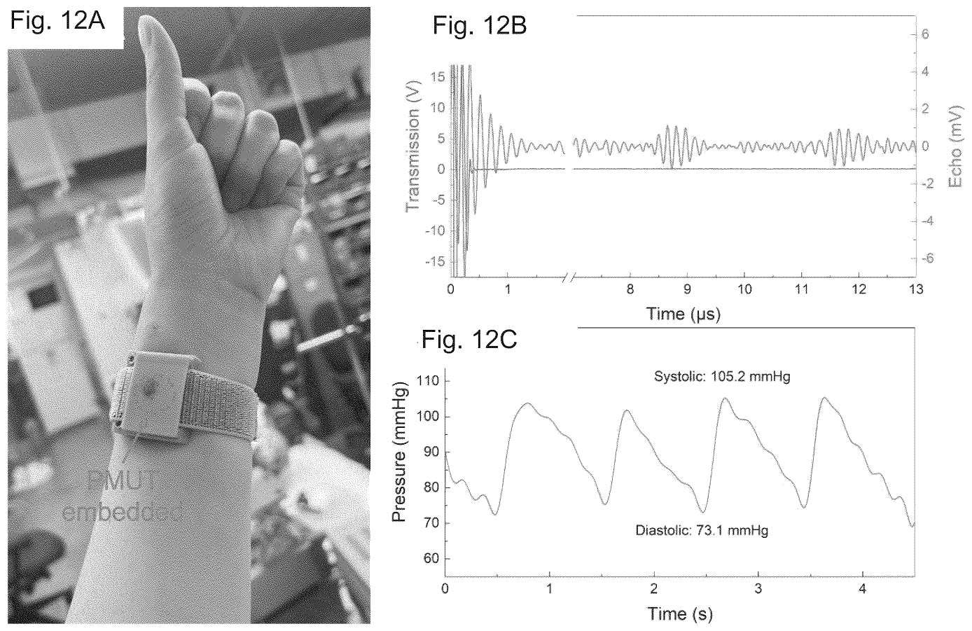

We have also successfully demonstrated integrating this device into non-invasive wearable forms. As depicted in FIG. 12A, the PMUT sensor is discreetly embedded beneath a 3D-printed wearable mold, simulating the form factor of a typical smartwatch. The sensor array is positioned in proximity to the radial artery, maintaining reliable contact with the volunteer through the watch band. FIG. 12B shows the echo signals where the echoes from the radial arteries can be clearly identified. The extracted diameter measures approximately 2.3 mm, closely aligning with real-world conditions. Following the dynamic schematic before, we were able to capture the blood pressure waveform of the volunteer, revealing a systolic pressure of 105.2 mmHg and a diastolic pressure of 73.1 mmHg (as shown in FIG. 12C). These values are consistent with the blood pressure measurements obtained using a traditional cuff.

### REFERENCES

[1] CDC: Health Topics—High Blood Pressure.
[2] Hong, Kuen Sik. Journal of Stroke, 2017, 19(2): 152-165.
[3] Kawasaki, Takeshi, et al. Cardiovascular research 21.9 (1987): 678-687.
[4] Akhbari, Sina et al., Journal of Microelectromechanical Systems, vol. 25, no. 2, pp. 326-336, 2016.

1. A blood pressure monitoring device comprising an array of piezoelectric micromachined ultrasonic transducers (PMUTs) configured for blood pressure monitoring.

2. The device of claim 1, further comprising both an active sensor and a related analog front-end (AFE) for wireless communications, configured to record and identify abnormal behaviors in real time.

3. The device of claim 1, configured for acoustic beamforming and line-scanning, so as to increase the stability and signal strength from noise sources.

4. A device herein, wherein beamforming technology is utilized to optimize ultrasonic energy, and signals measured by the PMUT sensor are analyzed and post-processed in an analog front-end (AFE).

5. The device of claim 1, configured as a wearable system composed of the PMUT array in a flexible substrate as the sensor, wherein beamforming optimizes ultrasonic energy, and signals measured by the PMUT sensor are analyzed and post-processed in a analog front-end (AFE) and transmitted to a portable device to monitor their blood pressure in real time.

6. The device of claim 1, configured as a 31× 35 array design and a total sensor size of 5 mm by 5 mm, wherein the radius of each element is 29 μm on a two 1-μm-thick AlN bimorph and dual-electrode diaphragm to have a designed frequency of 6 MHz in liquid, and the array structure has 20 independent channels for the purpose of beamforming.

7. The device of claim 1, configured as a 31×35 array design and a total sensor size of 5 mm by 5 mm, wherein the radius of each element is 29 μm on a two 1-μm-thick AlN bimorph and dual-electrode diaphragm to have a designed frequency of 6 MHz in liquid, and the array structure has 20 independent channels for the purpose of beamforming, as shown in FIGS. 2A-C.

8. The device of claim 1, configured as follows: a 200-nm seed AlN layer is first deposited by the AC sputtering process for good crystallinity, followed by the bottom Mo/bottom AlN/mid Mo depositions with the thickness of 150 nm, 1 μm and 150 nm, respectively; the middle Mo layer is then patterned, and a 1-μm thick top AlN layer and a 150-nm thick Mo electrode layer are deposited; the AlN layers are then patterned via the reactive ion etching process, wherein the diaphragm size is defined by a backside DRIE process, and the PMUT sensors are then connected to the outside circuit through wire bonding using an operation scheme.

9. The device of claim 1, configured as follows: a 200-nm seed AlN layer is first deposited by the AC sputtering process for good crystallinity, followed by the bottom Mo/bottom AlN/mid Mo depositions with the thickness of 150 nm, 1 μm and 150 nm, respectively; the middle Mo layer is then patterned, and a 1-μm thick top AlN layer and a 150-nm thick Mo electrode layer are deposited; the AlN layers are then patterned via the reactive ion etching process, wherein the diaphragm size is defined by a backside DRIE process, and the PMUT sensors are then connected to the outside circuit through wire bonding using an operation scheme, as shown in FIGS. 3A-F.

10. The device of claim 1, configured to deploy beamforming and use the phase-delay on elements/channels in the array such that the signal emitted by different elements/channels can be in phase at the focal points/lines by adding up the acoustic pressure, wherein different phase delays are applied to the system by adjusting the phase in PMUT sensors locating in different positions.

11. The device of claim 1, configured to deploy beamforming and use the phase-delay on elements/channels in the array such that the signal emitted by different elements/channels can be in phase at the focal points/lines by adding up the acoustic pressure, wherein different phase delays are applied to the system by adjusting the phase in PMUT sensors locating in different positions, as shown in FIGS. 4A-C.

12. The device of claim 1, configured to addresses the shift of the artery and sensor positions due to the motion of muscles/tissues, wherein the main reflected acoustic beam of the cylindrical artery comes from the path that follows the law of reflection; however, the main reflection path may change correspondingly if the artery shifts.

13. The device of claim 1, configured to addresses the shift of the artery and sensor positions due to the motion of muscles/tissues, wherein the main reflected acoustic beam of the cylindrical artery comes from the path that follows the law of reflection; however, the main reflection path may change correspondingly if the artery shifts, as shown in FIGS. 5A-B.

14. The device of claim 1, configured for a two-step method combining the adjustment and line-scanning beamforming method using each measurement to obtain the high signal outputs, wherein the transmitted arrays are controlled with different phases to achieve beamforming effect along the pre-defined path and the largest signal collected is used for the post-processing, to obtain signals coming from the right path and maintain the good signal-to-noise ratio.

15. The device of claim 1, comprising a horizontal section comprising multiple individually controlled arrays, wherein depending on the relative position of the artery, each array will receive distinct echo signals, wherein this information is used to fine-tune the device's position within the range where the primary vertical detection array, can precisely measure the required metrics, facilitated by beamforming techniques.

16. The device of claim 1, comprising a horizontal section comprising multiple individually controlled arrays, wherein depending on the relative position of the artery, each array will receive distinct echo signals, wherein this information is used to fine-tune the device's position within the range where the primary vertical detection array, can precisely measure the required metrics, facilitated by beamforming techniques, as shown in FIG. 6.

17. The device of claim 1, bonded to a printed circuit board (PCB) and comprising a defined pattern of a dual-electrode design providing crystallinity of the AlN with a pillar-like morphology.

18. The device of claim 1, bonded to a printed circuit board (PCB) and comprising a defined pattern of a dual-electrode design providing crystallinity of the AlN with a pillar-like morphology, as shown in FIGS. 7A-D.

19. The device of claim 1, integrated into a non-invasive wearable form; particularly wherein the PMUT sensor is discreetly embedded beneath a 3D-printed wearable mold, simulating the form factor of a typical smartwatch, the sensor array is positioned in proximity to the radial artery, maintaining reliable contact with a person through a watch band.

20. A method comprising monitoring blood pressure in real time with a device of claim 1, particularly wherein beamforming technology is utilized to optimize ultrasonic energy, and signals measured by the PMUT sensor are analyzed and post-processed in an analog front-end (AFE).
